(19) United States
(12) Patent Application Publication
(10) Pub. No.: US 2009/0270714 A1
(43) Pub. Date: Oct. 29, 2009

(54) Device to Treat Chronic Total Occlusions Using Energy Imparted By External Imaging

(75) Inventors: Niall Duffy, Ballybrit (IE); Michael Neidert, Ballybrit (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc, Santa Rosa, CA (US)

(21) Appl. No.: 12/108,921

(22) Filed: Apr. 24, 2008

(51) Int. Cl. A61B 5/05; A61B 8/12

(52) U.S. Cl. 600/411; 600/439

(57) ABSTRACT

A visualization and treatment system for treating a chronic total occlusion. The system includes an elongated member configured to be tracked to the chronic total occlusion. The elongated member has a transducer located at a distal end of the elongated member. The transducer is constructed and arranged to convert a first form of energy into a second form of energy to treat the chronic total occlusion. The system also includes an external imaging system constructed and arranged to create an image of the chronic total occlusion and to provide the first form of energy to the transducer.

### BACKGROUND OF THE INVENTION

1. Field of the Invention

The present invention is generally related to a device for treating chronic total occlusions in a vessel, particularly in a vessel located below the knee.

2. Background of the Invention

Stenotic lesions may comprise a hard, calcified substance and/or a softer thrombus material, each of which forms on the lumen walls of a blood vessel and restricts blood flow there through. Intra-luminal treatments such as balloon angioplasty (PTA, PTCA, etc.), stent deployment, atherectomy, and thrombectomy are well known and have proven effective in the treatment of such stenotic lesions. These treatments often involve the insertion of a therapy catheter into a patient's vasculature, which may be tortuous and may have numerous stenoses of varying degrees throughout its length. In order to place the distal end of a catheter at the treatment site, a guidewire is typically introduced and tracked from an incision, through the vasculature, and across the lesion. Then, a catheter (e.g. a balloon catheter), perhaps containing a stent at its distal end, can be tracked over the guidewire to the treatment site. Ordinarily, the distal end of the guidewire is quite flexible so that it can be rotatably steered and pushed through the bifurcations and turns of the typically irregular passageway without damaging the vessel walls.

In some instances, the extent of occlusion of the lumen is so severe that the lumen is completely or nearly completely obstructed, which may be described as a total occlusion. If this occlusion persists for a long period of time, the lesion is referred to as a chronic total occlusion or CTO. Furthermore, in the case of diseased blood vessels, the lining of the vessels may be characterized by the prevalence of atheromatous plaque, which may form total occlusions.

The extensive plaque formation of a chronic total occlusion typically has a fibrous cap surrounding softer plaque material. This fibrous cap may present a surface that is difficult to penetrate with a conventional guidewire, and the typically flexible distal tip of the guidewire may be unable to cross the lesion. Thus, for treatment of total occlusions, stiffer guidewires have been employed to recanalize through the total occlusion. However, due to the fibrous cap of the total occlusion, a stiffer guidewire still may not be able to cross the occlusion. When using a stiffer guidewire, great care must be taken to avoid perforation of the vessel wall.

Further, in a CTO, there may be a distortion of the regular vascular architecture such that there may be multiple small non-functional channels throughout the occlusion rather than one central lumen for recanalization. Thus, the conventional approach of looking for the single channel in the center of the occlusion may account for many of the failures. These spontaneously recanalized channels may be responsible for failures due to their dead-end pathways and misdirecting of the guidewires. Once a “false” tract is created by a guidewire, subsequent attempts with different guidewires may continue to follow the same incorrect path, and it is very difficult to steer subsequent guidewires away from the false tract.

Current CTO treatment typically uses fluoroscopy and angiography to visualize the vessels. However, because contrast cannot cross a CTO, it may be difficult to visualize the lesion and the portion of the vessel that is distal to the lesion. This may further impede efforts to cross the lesion.

Another equally important failure mode, even after a guidewire successfully crosses a chronic total occlusion, is the inability to advance a balloon or other angioplasty equipment over the guidewire due to the fibrocalcific composition of the chronic total occlusion, mainly both at the “entry” point and at the “exit” segment of the chronic total occlusion. Even with balloon inflations throughout the occlusion, many times there is no antegrade flow of contrast injected, possibly due to the recoil or insufficient channel creation throughout the occlusion.

Atherosclerotic plaques vary considerably in their composition from site to site, but certain features are common to all of them. They contain many cells; mostly these are derived from cells of the wall that have divided wildly and have grown into the surface layer of the blood vessel, creating a mass lesion. Plaques also contain cholesterol and cholesterol esters, commonly referred to as fat. This lies freely in the space between the cells and in the cells themselves. A large amount of collagen is present in the plaques, particularly advanced plaques of the type which cause clinical problems. Additionally, human plaques contain calcium to varying degrees, hemorrhagic material including clot and grumous material composed of dead cells, fat and other debris. Relatively large amounts of water are also present, as is typical of all tissue.

Successful recanalization of chronic total occlusions remains an area where improvements are needed. Approximately 30% of all coronary angiograms in patients with coronary artery disease will show a CTO and its presence often excludes patients from treatment by percutaneous coronary intervention. Acute success rates vary according to the duration of occlusion, the morphology of the lesion and the coronary anatomy, the experience of the operator, the degree of persistence employed, and the type of equipment used. Recanalization rates range between 45-80%, with the highest success in short, recently occluded (<1 month), non-calcified lesions.

It is desirable to be able to use energy imparted by an external imaging system and translate the energy to treat (e.g. soften or ablate) the CTO.

### SUMMARY OF THE INVENTION

The present invention describes an apparatus and method to treat a chronic total occlusion by using energy that is imparted to an elongated member, such as a catheter, by an external imaging system. Embodiments of the apparatus and method described herein may be particularly suited for treating occlusions that are located in peripheral arteries below the knee.

According to an aspect of the present invention, there is provided a visualization and treatment system for treating a chronic total occlusion. The system includes an elongated member configured to be tracked to the chronic total occlusion. The elongated member has a transducer located at a distal end of the elongated member. The transducer is constructed and arranged to convert a first form of energy into a second form of energy to treat the chronic total occlusion. The system also includes an external imaging system constructed and arranged to create an image of the chronic total occlusion and to provide the first form of energy to the transducer.

According to an aspect of the invention, there is provided a treatment device for a chronic total occlusion. The treatment device includes an elongated member configured to be tracked to the chronic total occlusion. The elongated member has a transducer located at a distal end of the elongated member. The transducer is configured to receive a first type of energy from an external imaging system and to convert the first type of energy into a second type of energy to treat the chronic total occlusion.

According to an aspect of the invention, there is provided a method for treating a chronic total occlusion in a vessel. The method includes inserting an elongated member into the vessel to a location adjacent to the chronic total occlusion. The elongated member has a transducer located at a distal end of the elongated member. The method also includes generating a first type of energy with an external imaging system, directing the first type of energy towards the transducer, and converting the first type of energy with the transducer into a second type of energy. The method also includes delivering the second type of energy to the chronic total occlusion to soften or ablate the chronic total occlusion.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIG. 1 is a schematic diagram of a vessel with a CTO;

FIG. 2 is a schematic diagram of visualization and treatment system according to an embodiment of the invention;

FIG. 3 is a schematic diagram of an embodiment of an elongated member of the visualization and treatment system of FIG. 2 within the vessel of FIG. 1; and

FIG. 4 is a schematic diagram of an embodiment of an elongated member of the visualization and treatment system of FIG. 2 within the vessel of FIG. 1.

### DETAILED DESCRIPTION OF EMBODIMENTS OF THE INVENTION

The following detailed description is merely exemplary in nature and is not intended to limit the invention or the application and use of the invention. Furthermore, there is no intention to be bound by any expressed or implied theory presented in the preceding technical field, background, brief summary or the following detailed description.

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

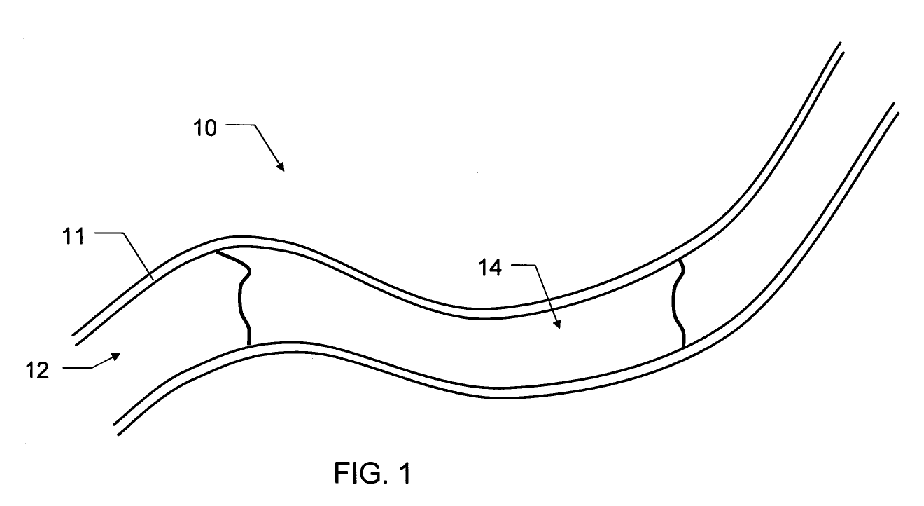

FIG. 1 illustrates a vessel 10 having an inner wall 11 that defines a lumen 12, and a chronic total occlusion (CTO) 14 within the lumen 12. The CTO 14 substantially or completely blocks flow of blood through the vessel 10. In an embodiment, the vessel 10 is a peripheral artery that is located below the patient's knee, i.e. between the knee and the foot of a patient's leg.

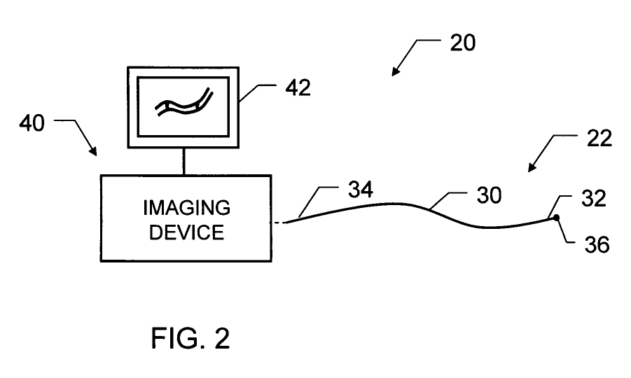

FIG. 2 illustrates a visualization and treatment system 20 according to an embodiment of the present invention. As illustrated, the visualization and treatment system 20 generally includes a treatment device 22 and an imaging device 40. The treatment device 22 includes an elongated member 30, which may be in the form of a catheter or a guidewire. The elongated member 30 has a distal end 32 that is configured to enter the lumen 12 of the vessel 10 and be tracked to the CTO 14 in a controlled manner by the clinician. The elongated member 30 also has a proximal end 34 that is configured to stay outside of the patient and be handled by the clinician.

The proximal end 34 of the elongated member 30 is connected to the imaging device 40. The imaging device 40 may be a magnetic resonance imaging (“MRI”) system, an electromagnetic navigation system, an ultrasonic imaging system, such as sonographic machine, or any other suitable imaging device that is commonly used in surgical procedures. Such visualization equipment is known in the art and therefore is not discussed in greater detail herein.

The elongated member 30 also includes a transducer 36 located at the distal end 32. The term “transducer” as used herein is defined as a device that is configured to convert one form of energy into another form of energy, as explained in further detail below with respect to embodiments of the invention. For example, the transducer may be a coil that converts the energy of a magnetic field into an electric current (and heat) or that converts an electrical current into a magnetic field. As another non-limiting example, the transducer may be a material that can be excited by waves of energy, such as acoustic or ultrasonic waves of energy, and convert the waves of energy into mechanical energy by vibration.

As illustrated by FIG. 2, the visualization and treatment system 20 also includes an output device 42 that is configured to output an image of the vessel 10 that is created by the imaging device 40. In an embodiment, the output device 42 may be a monitor that is configured to display the image of the vessel 10 and CTO 14, as illustrated by FIG. 2. The clinician operating the visualization and treatment system 20 may use the image to manipulate the elongated member 30 in the lumen 12 of the vessel 10. The output device 42 may also be configured to provide information on the condition and status of the treatment device 22, such as whether the transducer 36 is actively converting energy and/or an indication of the energy being output by the transducer 36. For example, in an embodiment wherein the transducer 36 is a coil, a thermocouple may be mounted to the distal end 32 of the elongated member 30. The thermocouple may be configured to provide a signal to the output device 42 to relay temperature information as the coil is heated in response to an externally applied magnetic field.

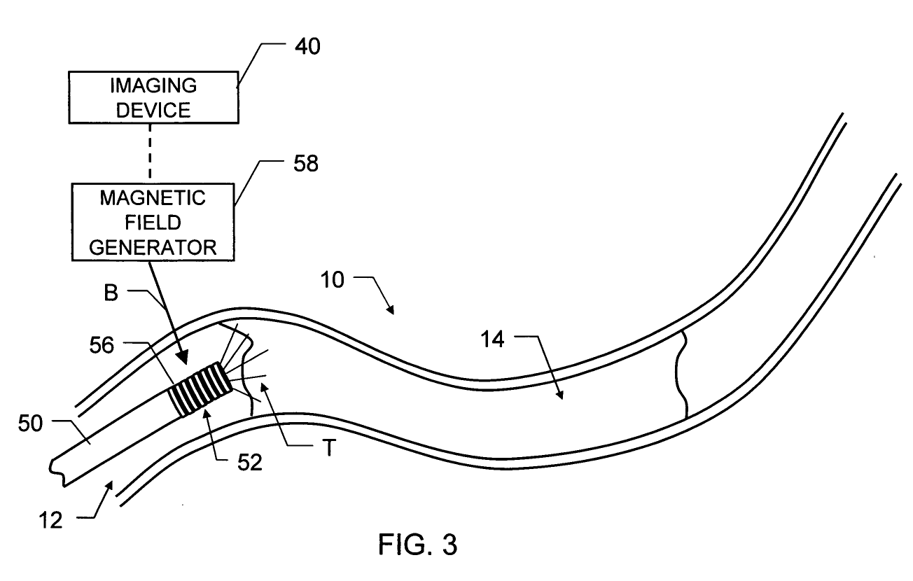

FIG. 3 illustrates an embodiment of the treatment device 22 in which the elongated member 30 is in the form of a catheter 50 that includes a distal end 52, and the transducer 36 is in the form of a coil 56 located at the distal end 52. The coil 56 may be constructed and arranged so that the catheter 50 is visible when using an external magnetic imaging system as the imaging device 40, such as an MRI system. Examples of such MRI systems include, but are not limited to systems from GE Medical Systems, Waukesha, Wis.; Siemens Medical Solutions of Siemens AG, Malvern, Pa. and Erlangen, Germany; and Toshiba America Medical Systems, Inc., Tustin, Calif. In an embodiment, the external imaging system may include an electromagnetic navigation system, which is also known in the art.

In an embodiment, the coil 56 may be considered to be a receiver coil that is configured to be detected by the imaging device 40 (in this embodiment an MRI system or an electromagnetic navigation system) and displayed on the output device 42. This enables the clinician to determine, from the display, when the distal end 52 of the catheter 50 has reached the CTO 14 within the vessel 10. The coil 56 may also be used by the imaging device 40 to create an image of the CTO 14 itself and of a portion of the vessel 10 that is distal to the CTO 14.

In an embodiment, a plurality of coils may be provided so as to provide rings or bands of windings along the distal portion of the catheter or along the entire length of the catheter to further enhance the image displayed on the output device 42. The illustrated embodiment is not intended to be limiting in any way.

The coil 56 may be made of a conductive material that is shielded along the majority of its length to inhibit interference, as is known in the art. Although a coil is illustrated in FIG. 3, it should be understood that other devices to create an image in an MRI or electromagnetic navigation system may also be used, including objects which may not be literally a coil. The coil 56 is connected to a proximal connector located at the proximal end of the catheter 50. The proximal connector may be connected to the imaging device 40 through an impedance matching circuit, as is known in the art, such that signals from the coil 56 are received by the imaging device 40.

The imaging device 40 may be programmed to display, in response to such signals received from the coil 56, the position of the coil 56 relative to anatomical structures within the patient's body in a so-called real time manner. In an embodiment, the image generated by the imaging device 40 may be superimposed into a prior MRI imaging scan, or even an image created by fluoroscopy, that is stored in memory of the imaging device 40 using known techniques.

As illustrated in FIG. 3, once the distal end 52 of the catheter 50 is located at the CTO 14, as determined by clinician using the imaging device 40, a magnetic field B provided by the imaging device 40 may be used create a current in the coil 56. The magnetic field B may be generated by a magnetic field generator 58, which may or may not be part of the imaging device 40. In an embodiment, the magnetic field generator 58 is not part of the imaging device 40. The current in the coil 56 that is created by the energy in the magnetic field B may generate thermal energy T (i.e., heat) that can be radiated and convected from the coil 56 to the CTO 14.

In an embodiment, the imaging device 40 may be programmed to deliver a magnetic field B that is strong enough to allow the coil 56 be heated to a suitable level that allows the coil 56 to deliver thermal energy T to the CTO 14, as illustrated in FIG. 3. The thermal energy T may be at a level that may result in the destruction of, for example, fibrine bonds of the plaque, thereby resulting in a softening or at least a partial opening of the vessel 10. By using the transducer 36, such as the coil 56, as an intermediary, the energy imparted by the imaging device 40, such as the MRI system, may be used to treat the CTO 14.

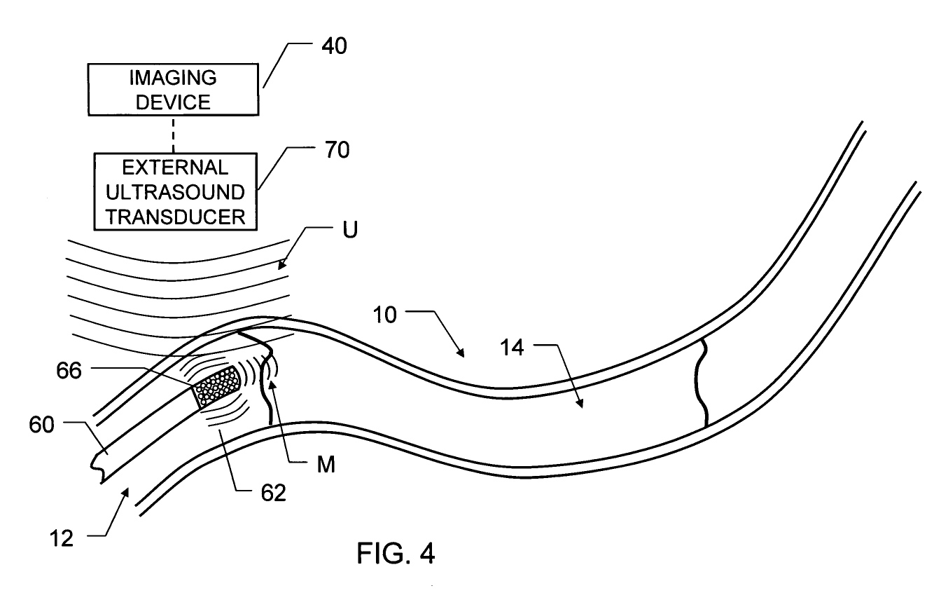

FIG. 4 illustrates an embodiment of the treatment device 22 in which the elongated member 30 is in the form of a catheter 60 that has a distal end 62, and the transducer 36 is in the form of acoustically activated material 66 located at the distal end 62. The acoustically activated material 66 may include a plurality of micro beads or any other suitable particles of material that may be packed together at the distal end 62 of the catheter 60. The acoustically activated material 66 is constructed and arranged to vibrate at a high frequency, i.e., resonate, when excited by energy waves, such as acoustic or ultrasonic waves. In an embodiment, the acoustically activated material 66 is packed at suitable density that allows the distal end 62 of the catheter 60 to provide mechanical energy to the CTO 14 when the acoustically activated material 66 vibrates or becomes activated.

In the embodiment illustrated by FIG. 4, an external ultrasound transducer 70, which converts electricity to ultrasonic energy waves, may be used to create images of the CTO 14 and surrounding tissue. This may allow the clinician to track the catheter 60 to the CTO 14. The external ultrasound transducer 70 may or may not be part of the imaging device 40. In an embodiment, the external ultrasound transducer 70 is separate from the imaging device 40 and is not used to create images of the CTO 14 and surrounding tissue.

The external ultrasound transducer 70 may be configured to generate ultrasonic energy waves U towards the acoustically activated material 66 at a frequency and amplitude that causes the acoustically activated material 66 to vibrate. The vibration created by the movement of the acoustically activated material 66 creates mechanical energy M that may be used to treat the CTO 14. The mechanical energy M may result in the destruction of, for example, fibrine bonds of the plaque, thereby resulting in at least a partial opening of the vessel 10.

After the CTO 14 has been treated by the treatment device 22, the clinician may be able to cross that CTO 14 and provide further types of treatment that are known in the art, if desired. Although embodiments of the invention as described herein provide treatment to the CTO 14 while the treatment device 22 is located outside and adjacent to the CTO 14, the treatment device 22 may be used to treat the CTO 14 while having the transducer 36 located within the CTO 14. The illustrated embodiments are not intended to be limiting in any way.

MRI and ultrasound may be used to visualize the CTO 14 and a portion of the vessel 10 that is distal to the CTO 14. Therefore, in embodiments of the invention, the catheters 50, 60 may be used to both treat the CTO 14, as described above, as well as assist the clinician to navigate the catheters 50, 60 to and through the CTO 14.

In an embodiment, an imaging device that is separate from the treatment device 22 may be used. For example, a fluoroscope may be used to create the image of the vessel 10 and CTO 14 using known methods, while a separate external energy device, such as the external magnetic field generator 58 or the external ultrasound transducer 70, may be used with the treatment device 22 to treat the CTO 14. The elongated member 30 may include a radiopaque material in the form of a marker band located at the distal end 32 to assist with the tracking of the elongated member 30 to the CTO 14. Radiopaque materials do not allow certain wavelengths of radiation, such as x-rays, to pass through, which allows the clinician to see the radiopaque material in a human body when using suitable visualization equipment, such as a fluoroscope. Examples of such radiopaque materials include, but are not limited to metals such as gold, platinum, and alloys thereof, and filled polymeric materials, such as barium sulfate loaded silicone, polyimide, and polycarbonate.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient roadmap for implementing an exemplary embodiment of the invention, it being understood that various changes may be made in the function and arrangement of elements described in an exemplary embodiment without departing from the scope of the invention as set forth in the appended claims.

What is claimed is:

1. A visualization and treatment system for treating a chronic total occlusion, the system comprising:
an elongated member configured to be tracked to the chronic total occlusion, the elongated member having a transducer located at a distal end thereof, the transducer being constructed and arranged to convert a first form of energy into a second form of energy to treat the chronic total occlusion; and
an external imaging system constructed and arranged to create an image of the chronic total occlusion and to provide the first form of energy to the transducer.

2. A visualization and treatment system according to claim 1, wherein the elongated member comprises a catheter.

3. A visualization and treatment system according to claim 1, wherein the transducer comprises a coil constructed and arranged to become heated when exposed to a magnetic field.

4. A visualization and treatment system according to claim 3, wherein the external imaging system comprises a magnetic resonance imaging system.

5. A visualization and treatment system according to claim 3, wherein the external imaging system comprises an electromagnetic navigation system.

6. A visualization and treatment system according to claim 1, wherein the transducer comprises an acoustically activated material that is configured to resonate and deliver mechanical energy when exposed to ultrasonic waves.

7. A visualization and treatment system according to claim 6, wherein the external imaging system comprises an external ultrasound transducer.

8. A treatment device for a chronic total occlusion, the treatment device comprising:
an elongated member configured to be tracked to the chronic total occlusion, the elongated member having a transducer located at a distal end thereof, the transducer being configured to receive a first type of energy from an external imaging system and to convert the first type of energy into a second type of energy to treat the chronic total occlusion.

9. A treatment device according to claim 8, wherein the elongated member comprises a catheter.

10. A treatment device according to claim 8, wherein the transducer comprises a coil constructed and arranged to become heated when exposed to a magnetic field.

11. A treatment device according to claim 8, wherein the transducer comprises an acoustically activated material that is configured to resonate and deliver mechanical energy when exposed to ultrasonic waves.

12. A method for treating a chronic total occlusion in a vessel, the method comprising:
inserting an elongated member into the vessel to a location adjacent to the chronic total occlusion, the elongated member having a transducer located at a distal end thereof;
generating a first type of energy with an external imaging system;
directing the first type of energy towards the transducer;
converting the first type of energy with the transducer into a second type of energy; and
delivering the second type of energy to the chronic total occlusion to soften or ablate the chronic total occlusion.

13. A method according to claim 12, wherein said generating energy comprises generating a magnetic field with the external imaging system.

14. A method according to claim 13, wherein the external imaging system comprises a magnetic resonance imaging system.

15. A method according to claim 13, wherein the external imaging system comprises an electromagnetic navigation system.

16. A method according to claim 13, wherein said transducer comprises a coil and said second energy comprises heat.

17. A method according to claim 12, wherein said generating energy comprises generating ultrasonic waves of energy with the external imaging system.

18. A method according to claim 17, wherein the external imaging system comprises an ultrasound transducer.

19. A method according to claim 17, wherein said transducer comprises acoustically activated material that resonates when exposed to the ultrasonic waves of energy and said second energy comprises mechanical energy.
